(19) United States
(12) Patent Application Publication
(10) Pub. No.: US 2007/0031353 A1
(43) Pub. Date: Feb. 8, 2007

(54) Cosmetic formulations comprising diketo diphenyl pyrrolo-pyrrole pigments

(76) Inventor: Olof Wallquist, Therwil (CH)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/575,513

(22) PCT Filed: Oct. 8, 2004
(86) PCT No.: PCT/EP04/52474

§ 371 Date: Apr. 13, 2006

(30) Foreign Application Priority Data
Oct. 17, 2003 (EP) 03103855.7

(51) Int. Cl. A61K 8/49

(52) U.S. Cl. 424/59; 424/63; 424/61

(57) ABSTRACT

The present invention relates to cosmetic formulations comprising at least one pigment of formula (I) wherein all substituents have the meanings as defined in the claims, and wherein the pigments have a specific surface area (BET) of 15-200 m²/g, as well as to compositions for making up the skin, both of the face and of the human body, keratinous fibers or superficial body growths, such as the nails, eyelashes, eyebrows or hair, and the lips.

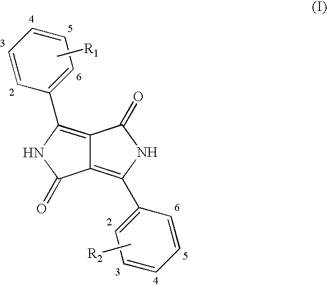

The present invention relates to cosmetic formulations comprising at least one pigment with an intense and saturated colour, more especially, to compositions for making up the skin, both of the face and of the human body, keratinous fibers or superficial body growths, such as the nails, eyelashes, eyebrows or hair, and the lips.

Makeup compositions, such as free or compacted powders, foundations, face powders, eyeshadows, lipsticks, products for concealing rings under the eyes, blushers, mascaras, eyeliners, lip pencils, eyeliner pencils, nail varnishes and products for making up the body are composed of an appropriate vehicle and of colouring agents of different natures intended to confer a certain colour on these compositions before and/or after their application to the skin, lips and/or superficial body growths.

These colouring agents can be lakes, inorganic or organic pigments and/or pearlescent pigments or alternatively colorants. Cosmetic scientists have available pigments of inorganic origin, such as iron oxides or mixtures of brown-yellow iron oxides, and pigments of organic origin. Inorganic pigments, in particular inorganic oxides, have the advantage of being very stable but have the disadvantage of giving rather drab and pale colours. Organic lakes have the advantage of conferring more saturated colours on the compositions but the majority is unstable with respect to light, temperature or pH. Some of these lakes also exhibit the disadvantage of staining the skin in an unsightly way after application, by escape of the colorant. Pearlescent pigments, for their part, make it possible to obtain varied but never intense colours with effects which are iridescent but which are generally fairly weak.

Therefore, there is still a need for further formulations having improved colour strength and saturated shades which, in addition to good tolerability, additionally exhibit outstanding fastness properties in as many areas as possible.

It has now been found that the formulations according to the invention meet those requirements.

By virtue of the pigments used according to the invention in the novel formulations, it is possible to obtain colour shades that hitherto were difficult to achieve or could not be achieved at all. The new pigment formulations have outstanding pigmentary properties, such as lightfastness, chroma/saturation, colour strength, hiding power and dispersibility. Further, the colour is almost identical with the colour that can be achieved on the skin and nails using the novel formulations. As a result it is very readily possible to achieve precisely the desired colour shade. The novel formulations are distinguished especially by the fact that they exhibit no “bleeding” of the pigments into the skin and the nails, that is to say sharp outlines are obtained therewith on the skin and the nails themselves are not stained.

An embodiment of the invention is a cosmetic formulation comprising at least one pigment of formula (I) 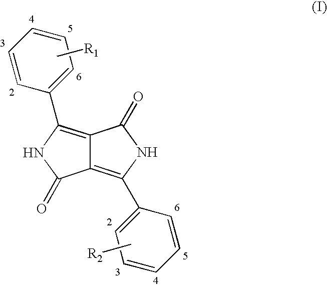
wherein
R₁ signifies hydrogen; SO₃M; linear or branched C₁-C₁₈alkyl, which can be unsubstituted or substituted by one or more halogen, OH, OR₃, SR₃, SO₂NR₃R₄, NH₂, NR₃R₄, COOR₄, CONR₃R₄, OCOR₃ or SO₃M; linear or branched C₁-C₄alkoxy, which can be unsubstituted or substituted by one or more halogen, OH, OR₃, SR₃, SO₂NR₃R₄, NH₂, NR₃R₄, COOR₄, CONR₃R₄, OCOR₃ or SO₃M; halogen; phenyl, which can be unsubstituted or substituted by one or more halogen, OH, OR₃, SR₃, SO₂NR₃R₄, NH₂, NR₃R₄, COOR₄, CONR₃R₄, OCOR₃ or SO₃M,
R₂ signifies hydrogen; SO₃M; linear or branched C₁-C₁₈alkyl, which can be unsubstituted or substituted by one or more halogen, OH, OR₃, SR₃, SO₂NR₃R₄, NH₂, NR₃R₄, COOR₄, CONR₃R₄, OCOR₃ or SO₃M; linear or branched C₁-C₄alkoxy, which can be unsubstituted or substituted by one or more halogen, OH, OR₃, SR₃, SO₂NR₃R₄, NH₂, NR₃R₄, COOR₄, CONR₃R₄, OCOR₃ or SO₃M; halogen; phenyl, which is substituted by one or more halogen, OH, OR₃, SR₃, SO₂NR₃R₄, NH₂, NR₃R₄, COOR₄, CONR₃R₄, OCOR₃ or SO₃M, wherein
   R₃ signifies linear or branched C₁-C₃₀alkyl; C₃-C₃₀-alkenyl; C₃-C₁₂cycloalkyl; C₆-C₁₄aryl, which can be unsubstituted or substituted by one or more C₁-C₆alkyl, C₅-C₆cycloalkyl, C₁-C₆alkoxy, C₁-C₆thioalkyl or halogen; C₇-C₂₄aralkyl, which can be unsubstituted or substituted by one or more C₁-C₆alkyl, C₅-C₆cycloalkyl, C₁-C₆alkoxy, C₁-C₆thioalkyl or halogen or C₈-C₂₄aralkenyl, which can be unsubstituted or substituted by one or more C₁-C₆alkyl, C₅-C₆cycloalkyl, C₁-C₆alkoxy, C₁-C₆thioalkyl or halogen; or C₁-C₄alkylene-C₄-C₈cylcoalkyl, wherein the ring contains at least one heteroatom selected from the group of N, O or S,
   R₄ signifies hydrogen; linear or branched C₁-C₃₀alkyl; C₃-C₃₀-alkenyl; C₃-C₁₂cycloalkyl; C₆-C₁₄aryl, which can be unsubstituted or substituted by one or more C₁-C₆alkyl, C₅-C₆cycloalkyl, C₁-C₆alkoxy, C₁-C₈thioalkyl or halogen; C₇-C₂₄aralkyl, which can be unsubstituted or substituted by one or more C₁-C₆alkyl, C₅-C₆cloalkyl, C₁-C₆alkoxy, C₁-C₆thioalkyl or halogen or C₈-C₂₄aralkenyl, which can be unsubstituted or substituted by one or more C₁-C₆alkyl, C₅-C₆cycloalkyl, C₁-C₆alkoxy, C₁-C₆thioalkyl or halogen; or C₁-C₄alkylene-C₄-C₈cylcoalkyl, wherein the ring contains at least one heteroatom selected from the group of N, O or S, and
M signifies hydrogen; a metal atom; or an unsubstituted or substituted ammonium group, wherein the pigments have a specific surface area (BET) of 15-200 m²/g, and with the proviso that if
   (i) R₁ is H or Cl in 4 position, then R₂ is not H or Cl in 4 position.

The specific surface area (BET) is measured according to the method developed by Brunauer, Emmet and Teller. The standardized method is described in DIN 66131 and DIN 66132.

Preferably, the pigments have a specific surface area (BET) of 20-200 m²/g, even more preferred of 25-200 m²/g.

Further preferred, the pigments have a specific surface area (BET) of 15-170 m²/g, more preferred of 15-150 m²/g.

Further more preferred, the pigments have a specific surface area (BEr) of 20-170 m²/g, even more preferred of 20-150 m²/g.

Especially preferred, the pigments have a specific surface area (BET) of 25-170 m²/g, very especially preferred of 25-150 m²/g.

The pigments according to the present invention have an average primary particle size of <0.2 μm. Preferably, the pigments according to the present invention have an average primary particle size <0.1 μm. More preferably, the pigments according to the present invention have an average primary particle size <0.2 μm and >0.01 μm, preferably >0.015 μm, even more preferably >0.02 μm.

Especially preferably, the pigments according to the present invention have an average primary particle size <0.1 μm and >0.01 μm, preferably >0.015 μm, even more preferably >0.02 μm.

The primary particle size is defined as the length of the longest dimension and it is estimated by the analysis of the transmission electron microscopy.

The alkyl and alkoxy radicals can be linear or branched and can be chosen for example, from methyl, ethyl, n- and isopropyl, n-, sec-, tert- or isobutyl, n-, sec-, tert- or isopentyl radicals; the alkenyl radicals can be linear or branched and can be chosen for example from allyl, methallyl, 2-butenyl, 2-hexenyl, 3-hexenyl or 2-octenyl radicals.

The alkyl chains can also interrupted by one or more heteroatoms, such as N, O or S.

The halogen atom can preferably be Cl, Br or F.

Preferably M is hydrogen, sodium, potassium, lithium or an unsubstituted or substituted ammonium group.

In a preferred embodiment of the present invention R₁ signifies hydrogen; linear or branched C₁-C₁₂alkyl, which can be unsubstituted or substituted by one or more halogen, OH, OR₃, SR₃, SO₂NR₃R₄, NH₂, NR₃R₄, COOR₄, CONR₃R₄, OCOR₃ or SO₃M; linear or branched C₁-C₄alkoxy, which can be unsubstituted or substituted by one or more halogen, OH, OR₃, SR₃, NH₂, SO₂NR₃R₄, NR₃R₄, COOR₄, CONR₃R₄, OCOR₃ or SO₃M; halogen; phenyl, which can be unsubstituted or substituted by one or more halogen, OH, OR₃, SR₃, SO₂NR₃R₄, NH₂, NR₃R₄, COOR₄, CONR₃R₄, OCOR₃ or SO₃M, wherein R₃, R₄ and M have the same meanings as defined above.

In a preferred embodiment of the present invention R₂ signifies hydrogen; linear or branched C₁-C₁₂alkyl, which can be unsubstituted or substituted by one or more halogen, OH, OR₃, SR₃, SO₂NR₃R₄, NH₂, NR₃R₄, COOR₄, CONR₃R₄, OCOR₃ or SO₃M; linear or branched C₁-C₄alkoxy, which can be unsubstituted or substituted by one or more halogen, OH, OR₃, SR₃, SO₂NR₃R₄, NH₂, NR₃R₄, COOR₄, CONR₃R₄, OCOR₃ or SO₃M; halogen; phenyl, which is substituted by one or more halogen, OH, OR₃, SR₃, NH₂, NR₃R₄, COOR₄, CONR₃R₄, OCOR₃ or SO₃M,

wherein R₃, R₄ and M have the same meanings as defined above.

In a more preferred embodiment of the present invention R₁ signifies hydrogen; linear or branched C₁-C₁₂alkyl, which can be unsubstituted or substituted by one or more halogen, OH, OR₃, SR₃, SO₂NR₃R₄, NH₂, NR₃R₄, COOR₄, CONR₃R₄, OCOR₃ or SO₃M; linear or branched C₁-C₄alkoxy, which can be unsubstituted or substituted by one or more halogen, OH, OR₃, SR₃, SO₂NR₃R₄, NH₂, NR₃R₄, COOR₄, CONR₃R₄, OCOR₃ or SO₃M; halogen; phenyl, which can be unsubstituted or substituted by one or more halogen, OH, ORE, SR₃, SO₂NR₃R₄, NH₂, NR₃R₄, COOR₄, CONR₃R₄, OCOR₃ or SO₃M, wherein R₃ signifies linear or branched C₁-C₁₈alkyl; C₃-C₁₈-alkenyl; C₃-C₈cycloalkyl; C₆-C₈aryl, which can be unsubstituted or substituted by one or more C₁-C₄alkyl, C₅-C₆cycloalkyl, C₁-C₄alkoxy, C₁-C₄thioalkyl or halogen; C₇-C₁₈aralkyl, which can be unsubstituted or substituted by one or more C₁-C₄alkyl, C₅-C₆cycloalkyl, C₁-C₄alkoxy, C₁-C₄thioalkyl or halogen or C₈-C₁₈aralkenyl, which can be unsubstituted or substituted by one or more C₁-C₄alkyl, C₅-C₆cloalkyl, C₁-C₄alkoxy, C₁-C₄thioalkyl or halogen; or C₁-C₄alkylene-C₄-C₈cylcoalkyl, wherein the ring contains at least one heteroatom selected from the group of N, O or S, R₄ signifies hydrogen; linear or branched C₁-C₁₈alkyl; C₃-C₁₈-alkenyl; C₃-C₈cycloalkyl; C₆-C₈aryl, which can be unsubstituted or substituted by one or more C₁-C₄alkyl, C₅-C₆cycloalkyl, C₁-C₄alkoxy, C₁-C₄thioalkyl or halogen; C₇-C₁₈aralkyl, which can be unsubstituted or substituted by one or more C₁-C₄alkyl, C₅-C₆cycloalkyl, C₁-C₄alkoxy, C₁-C₄thioalkyl or halogen or C₈-C₁₈aralkenyl, which can be unsubstitied or substituted by one or more C₁-C₄alkyl, C₅-C₆cycloalkyl, C₁-C₄alkoxy, C₁-C₄thioalkyl or halogen; or C₁-C₄alkylene-C₄-C₈cylcoalkyl, wherein the ring contains at least one heteroatom selected from the group of N, O or S, and

M signifies hydrogen; a metal atom; or an unsubstituted or substituted ammonium group.

In a more preferred embodiment of the present invention R₂ signifies hydrogen; linear or branched C₁-C₁₂alkyl, which can be unsubstituted or substituted by one or more halogen, OH, OR₃, SR₃, SO₂NR₃R₄, NH₂, NR₃R₄, COOR₄, CONR₃R₄, OCOR₃ or SO₃M; linear or branched C₁-C₄alkoxy, which can be unsubstituted or substituted by one or more halogen, OH, OR₃, SR₃, SO₂NR₃R₄, NH₂, NR₃R₄, COOR₄, CONR₃R₄, OCOR₃ or SO₃M; halogen; phenyl, which is substituted by one or more halogen, OH, OR₃, SR₃, SO₂NR₃R₄, NH₂, NR₃R₄, COOR₄, CONR₃R₄, OCOR₃ or SO₃M, wherein

R₃ signifies linear or branched C₁-C₁₈alkyl; C₃-C₁₈alkenyl; C₃-C₈cycloalkyl; C₆-C₈aryl, which can be unsubstituted or substituted by one or more C₁-C₄alkyl, C₅-C₆cycloalkyl, C₁-C₄alkoxy, C₁-C₄thioalkyl or halogen; C₇-C₁₈aralkyl, which can be unsubstituted or substituted by one or more C₁-C₄alkyl, C₅-C₆cycloalkyl, C₁-C₄alkoxy, C₁-C₄thioalkyl or halogen or C₈-C₁₈aralkenyl, which can be unsubstituted or substituted by one or more C₁-C₄alkyl, C₅-C₆cycloalkyl, C₁-C₄alkoxy, C₁-C₄thioalkyl or halogen; or C₁-C₄alkylene-C₄-C₈cylcoalkyl, wherein the ring contains at least one heteroatom selected from the group of N, O or S,

R₄ signifies hydrogen; linear or branched C₁-C₁₈alkyl; C₃-C₁₈-alkenyl; C₃-C₈cycloalkyl; C₆-C₈aryl, which can be unsubstituted or substituted by one or more C₁-C₄alkyl, C₅-C₆cycloalkyl, C₁-C₄alkoxy, C₁-C₄thioalkyl or halogen; C₇-C₁₈aralkyl, which can be unsubstituted or substituted by one or more C₁-C₄alkyl, C₅-C₆cycloalkyl, C₁-C₄alkoxy, C₁-C₄thioalkyl or halogen or C₈-C₁₈aralkenyl, which can be unsubstituted or substituted by one or more C₁-C₄alkyl, C₅-C₆cycloalkyl, C₁-C₄alkoxy, C₁-C₄thioalkyl or halogen; or C₁-C₄alkylene-C₄-C₈cylcoalkyl, wherein the ring contains at least one heteroatom selected from the group of N, O or S, and

M signifies hydrogen; a metal atom; or an unsubstituted or substituted ammonium group.

An especially preferred embodiment of the invention is a cosmetic formulation comprising at least one pigment of formula (I) 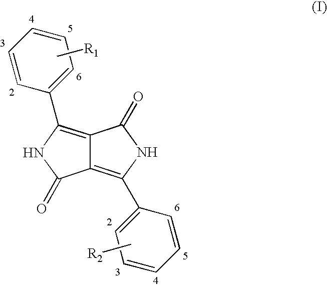
wherein
R₁ signifies hydrogen; linear or branched C₁-C₈alkyl, which can be unsubstihued or substituted by one or more OR₃, SR₃, SO₂NR₃R₄, NR₃R₄ or COOR₄; linear or branched C₁-C₄alkoxy, which can be unsubstituted or substituted by one or more OR₃, SR₃, SO₂NR₃R₄, NR₃R₄ or COOR₄; halogen; phenyl, which can be unsubstituted or substituted by one or more OR₃, SR₃, SO₂NR₃R₄, NR₃R₄ or COOR₄,
R₂ signifies hydrogen; linear or branched C₁-C₈alkyl, which can be unsubstituted or substituted by one or more OR₃, SR₃, SO₂NR₃R₄, NR₃R₄ or COOR₄; linear or branched C₁-C₄alkoxy, which can be unsubstituted or substituted by one or more OR₃, SR₃, SO₂NR₃R₄, NR₃R₄ or COOR₄; halogen; phenyl, which is substituted by one or more OR₃, SR₃, SO₂NR₃R₄, NR₃R₄ or COOR₄,
   wherein
   R₃ signifies linear or branched C₁-C₁₂alkyl; C₃-C₁₂-alkenyl; C₃-C₈cycloalkyl; C₆-C₈aryl, which can be unsubstituted or substituted by one or more C₁-C₄alkyl, C₅-C₆cycloalkyl, C₁-C₄alkoxy, C₁-C₄thioalkyl or halogen; C₇-C₁₂aralkyl, which can be unsubstituted or substituted by one or more C₁-C₄alkyl, C₅-C₆cycloalkyl, C₁-C₄alkoxy, C₁-C₄thioalkyl or halogen or C₈-C₁₂aralkenyl, which can be unsubstituted or substituted by one or more C₁-C₄alkyl, C₅-C₆cycloalkyl, C₁-C₄alkoxy, C₁-C₄thioalkyl or halogen; C₁-C₄alkylene-morpholino; or C₁-C₄alkylene-piperidino, and
   R₄ signifies hydrogen; linear or branched C₁-C₁₂alkyl; C₃-C₁₂-alkenyl; C₃-C₈cycloalkyl; C₆-C₆aryl, which can be unsubstituted or substituted by one or more C₁-C₄alkyl, C₆-C₆cycloalkyl, C₁-C₄alkoxy, C₁-C₄thioalkyl or halogen; C₇-C₁₂aralkyl, which can be unsubstituted or substituted by one or more C₁-C₄alkyl, C₅-C₆cycloalkyl, C₁-C₄alkoxy, C₁-C₄thioalkyl or halogen or C₈-C₁₂aralkenyl, which can be unsubstituted or substituted by one or more C₁-C₄alkyl, C₅-C₆cycloalkyl, C₁-C₄alkoxy, C₁-C₄thioalkyl or halogen; C₁-C₄alkylene-morpholino; or C₁-C₄alkylene-piperidino,
      wherein the pigments have a specific surface area (BET) of 15-200 m²/g, preferably of 20-200 m²/g, more preferably of 25-200 m²/g and with the proviso that if
   (i) R₁ is H or Cl in 4 position, then R₂ is not H or Cl in 4 position.

All preferences for the specific surface area as defined above also apply for the especially preferred embodiment of the invention.

A very especially preferred embodiment of the invention is a cosmetic formulation comprising at least one pigment of formula (I) 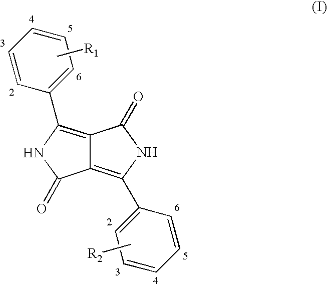
wherein
R₁ signifies hydrogen; linear or branched C₁-C₆alkyl, which can be unsubstituted or substituted by one or more OR₃, SR₃, SO₂NR₃R₄, NR₃R₄ or COOR₄; linear or branched C₁-C₄alkoxy, which can be unsubstituted or substituted by one or more OR₃, SR₃, SO₂NR₃R₄, NR₃R₄ or COOR₄; halogen; phenyl, which can be unsubstituted or substituted by one or more OR₃, SR₃, SO₂NR₃R₄, NR₃R₄ or COOR₄,
R₂ signifies hydrogen; linear or branched C₁-C₆alkyl, which can be unsubstituted or substituted by one or more OR₃, SR₃, SO₂NR₃R₄, NR₃R₄ or COOR₄; linear or branched C₁-C₄alkoxy, which can be unsubstituted or substituted by one or more OR₃, SR₃, SO₂NR₃R₄, NR₃R₄ or COOR₄; halogen; phenyl, which is substituted by one or more OR₃, SR₃, SO₂NR₃R₄, NR₃R₄ or COOR₄,
   wherein
   R₃ signifies linear or branched C₁-C₈alkyl; C₃-C₈alkenyl; C₃-C₈cycloalkyl; C₆-C₈aryl, which can be unsubstituted or substituted by one or more C₁-C₄alkyl, C₅-C₆cycloalkyl, C₁-C₄alkoxy, C₁-C₄thioalkyl or halogen; C₇-C₁₂aralkyl, which can be unsubstituted or substituted by one or more C₁-C₄alkyl, C₅-C₆cycloalkyl, C₁-C₄alkoxy, C₁-C₄thioalkyl or halogen or C₈-C₁₂aralkenyl, which can be unsubstituted or substituted by one or more C₁-C₄alkyl, C₅-C₆cycloalkyl, C₁-C₄alkoxy, C₁-C₄thioalkyl or halogen; C₁-C₄alkylene-morpholino; or C₁-C₄alkylene-piperidino and
   R₄ signifies hydrogen; linear or branched C₁-C₈alkyl; C₃-C₈-alkenyl; C₃-C₈cycloalkyl; C₆-C₈aryl, which can be unsubstituted or substituted by one or more C₁-C₄alkyl, C₅-C₆cycloalkyl, C₁-C₄alkoxy, C₁-C₄thioalkyl or halogen; C₇-C₁₂aralkyl, which can be unsubstituted or substituted by one or more C₁-C₄alkyl, C₅-C₆cycloalkyl, C₁-C₄alkoxy, C₁-C₄thioalkyl or halogen or C₈-C₁₂aralkenyl, which can be unsubstituted or substituted by one or more C₁-C₄alkyl, C₅-C₆cycloalkyl, C₁-C₄alkoxy, C₁-C₄thioalkyl or halogen; C₁-C₄alkylene-morpholino; or C₁-C₄alkylene-piperidino,
      wherein the pigments have a specific surface area (BET) of 15-200 m²/g, preferably, 20-200 m²/g, more preferably 25-200 m²/g and with the proviso that if
   (i) R₁ is H or Cl in 4 position, then R₂ is not H or Cl in 4 position.

All preferences for the specific surface area as defined above also apply for the very especially preferred embodiment of the invention.

The pigment of the invention may be transparent or opaque and can be a physical mixture or a solid solution or a mixed crystal of two or more pigments of the formula (I) or of pigments of the formula (I) with one or more of other organic pigments. The pigment of the invention may optionally be combined with other pigments for shifting the colour of the formulation or for enhancing the colour power and/or goniochromatic properties of crystal liquid or multilayer pigments having goniochromatic properties.

The manufacture of the diketodiarylpyrrolo-pyrroles of formula (I) is disclosed in particular in the Ciba-Geigy documents EP-A-94,911, EP-A-542,669, EP-A-787,730, EP-A-787,731 and WO-A-96/08537. The specific surface area (BET) and the average primary particle size can be controlled by commonly known methods, such as growth inhibitors, acid pasting, basic reprecipitation, mechanical methods, for example dry (salt) grinding (or milling), kneading, wet milling etc.

The pigment according to the invention may be incorporated in a cosmetic formulation, in an amount which can be easily determined by a person skilled in the art on the basis of his broad knowledge and which can in particular range from 0.01 to 50% by weight with respect to the weight of the formulation, preferably in an amount ranging from 0.5 to 25% by weight. This pigment can be also fixed on a polymer in particular by graphing or embedding. Moreover, it is also possible to use one or more pigments of formula (I) together with other pigments, goniochromatic pigments and/or colourants such as are employed in cosmetic formulations. Pigments other than those of formula (I) may be present in the formulation in an amount ranging from 0 to 25% of the weight of the final formulation and preferably from 2 to 15%. Preferred inorganic pigments are, for example, titanium, zirconium or cerium oxides, as well as zinc, iron or chromium oxides and ferric blue. Preferred organic pigments are, for example, carbon black and barium, strontium, calcium and aluminium lakes.

Further suitable pigments are those described in EP408498, EP953343 or WO0033795. If desired, the pigments may also be used in the form of surface-modified pigments, for example modified by perfluoroalkyl phosphate, methylpolysiloxanes, methyl-hydrogen-polysiloxanes or chitosan. Suitable modified pigments are, for example, those described by B. G. Hays in Am. Inkmaker, (June, 1984) 28, (October, 1986) 13 and (November, 1990) 28.

In addition, it is also possible to use solid solutions of the pyrrolo-[3,4-c]-pyrroles, for example solid solutions consisting of two different compounds of that type, such as are described in U.S. Pat. No. 4,783,540, or solid solutions of pyrrolo-[3,4-]-pyrroles and quinacridones, such as are described in U.S. Pat. No. 4,810,304, or solid solutions consisting of two different pyrrolo-[3,4-c]-pyrroles and quinacridones, such as are described in U.S. Pat. No. 5,529,623.

Such optionally modified pigments are advantageously used in the form of pigment preparations in which the pigment is already in dispersed form. Suitable preparations are described, for example, in W. Herbst, K. Hunger: Industrielle organische Pigmente, VCH Verlags-gesellschaft 1995, page 92 ff.

Therefore, a further embodiment of the present invention relates to a cosmetical formulation comprising, based on the total weight of the formulation,
a) from 0.0001 to 50% by weight, preferably from 0.0001 to 25% by weight, of at least one pigment of formula (I), and
b) from 50 to 99.9999% by weight, preferably from 75 to 99.9999% by weight, of a cosmetically suitable carrier.

Suitable carriers for the cosmetic preparations and formulations according to the invention are the conventional materials used in such compositions.

The cosmetic preparations and formulations according to the invention may be in the form of, for example, sticks, ointments, creams, emulsions, suspensions, dispersions, powders or solutions. They are, for example, lipsticks, mascara preparations, make-up for the cheeks, eyeshadows, foundations, eyeliners, powders or nail varnishes.

When the preparations are in the form of sticks, for example lipsticks, eyeshadows, make-up for the cheeks or foundations, such preparations consist for a considerable part of fatty components, which may consist of one or more waxes, for example ozocerite, lanolin, lanolin alcohol, hydrogenated lanolin, acetylated lanolin, lanolin wax, beeswax, candelilla wax, microcrystalline wax, carnauba wax, cetyl alcohol, stearyl alcohol, cocoa butter, lanolin fatty acids, petrolatum, petroleum jelly, mono-, di- or tri-glycerides or -fatty esters that are solid at 25° C., silicone waxes, such as methyloctadecane-oxypolysiloxane and poly(dimethylsiloxy)-stearoxysiloxane, stearic acid monoethanolamine, colophane and derivatives thereof, such as glycol abietates and glycerol abietates, hydrogenated oils that are solid at 25° C., sugar glycerides and oleates, myristates, lanolates, stearates and dihydroxy stearates of calcium, magnesium, zirconium and aluminium.

The fatty component may also consist of a mixture of at least one wax and at least one oil, in which case the following oils, for example, come into consideration: paraffin oil, purcellin oil, perhydrosqualene, sweet almond oil, avocado oil, calophyllum oil, castor oil, sesame oil, jojoba oil, mineral oils having a boiling point of approximately from 310 to 410° C., silicone oils, such as dimethylpolysiloxane, linoleic alcohol, linolenic alcohol, oleyl alcohol, cereal grain oils, such as wheatgerm oil, isopropyl lanolate, isopropyl palmitate, isopropyl myristate, butyl myristate, cetyl myristate, hexadecyl stearate, butyl stearate, decyl oleate, acetyl glycerides, octanoates and decanoates of alcohols and polyalcohols, for example of glycol and glycerol, ricinoleates of alcohols and polyalcohols, for example of cetyl alcohol, isostearyl alcohol, isocetyl lanolate, isopropyl adipate, hexyl laurate and octyldodecanol.

The fatty components in such preparations in the form of sticks may generally account for up to 99.9999% by weight of the total weight of the preparation.

The cosmetic preparations and formulations according to the invention may additionally comprise further constituents, for example glycols, polyethylene glycols, polypropylene glycols, monoalkanolamides, undyed polymeric, inorganic or organic fillers, preservatives, UV filters or other adjuvants and additives conventionally employed in cosmetics.

Such further constituents are, for example, a natural or a synthetic or a semi-synthetic di- or tri-glyceride, a mineral oil, a silicone oil, a wax, a fatty alcohol, a Guerbet alcohol or an ester thereof, a lipophilic functional cosmetic active ingredient including sunscreens, or a mixture of such substances.

A lipophilic functional cosmetic active ingredient suitable for skin cosmetics, an active ingredient combination or an active ingredient extract is an ingredient or a mixture of ingredients that is approved for dermal or topical application. The following may be mentioned by way of example:
active ingredients having a cleansing action on the skin surface and the hair. These include all substances that serve to cleanse the skin, such as oils, soaps, soapless detergents and solid substances;
active ingredients having a deodorizing and perspiration-inhibiting action: they include antiperspirants based on aluminium or zinc salts, deodorants comprising bactericidal or bacteriostatic deodorizing substances, for example tridosan, hexachlorophene, alcohols and cationic substances, for example quaternary ammonium salts, and odour absorbers, for example ®Grillocin (combination of zinc ricinoleate and various additives) or triethyl citrate, optionally in combination with an antioxidant, for example butyl hydroxytoluene) or ion-exchange resins;
active ingredients that offer protection against sunlight (UV filters): suitable active ingredients are filter substances (sunscreens) that are able to absorb W radiation from sunlight and convert it into heat. According to the desired action, the following light-protection agents are preferred: light-protection agents that selectively absorb sunburn-causing high-energy UV radiation in the range of approximately from 280 to 315 nm (UV-B absorbers) and transmit the longer-wave range of approximately from 315 to 400 nm (UV-A range), as well as light-protection agents that absorb only the longer-wave radiation of the UV-A range of from 315 to 400 nm (UV-A absorbers).
Suitable light-protection agents are, for example, organic UV absorbers from the class of the p-aminobenzoic acid derivatives, salicylic acid derivatives, benzophenone derivatives, dibenzoylmethane derivatives, diphenyl acrylate derivatives, benzofuran derivatives, polymeric UV absorbers comprising one or more organosilicon radicals, cinnamic acid derivatives, camphor derivatives, trianilino-s-triazine derivatives, phenylbenzimidazole sulfonic acid and salts thereof, menthyl anthranilates, benzotriazole derivatives, and/or an inorganic micropigment selected from aluminium oxide- or silicon dioxide-coated TiO₂, zinc oxide or mica;
active ingredients against insects (repellents): repellents are agents that are to prevent insects from touching the skin and becoming active there. They drive insects away and evaporate slowly. The most frequently used repellent is diethyl toluamide (DEET). Other common repellents will be found in VW. Raab and U. Kindl, “Pflegekosmetik”, Gustav-Fischer-Verlag Stuttgart/New York, 1991, p. 161;
active ingredients providing protection against chemical and mechanical influences: these include all substances that form a barrier between the skin and external harmful substances, for example paraffin oils, silicone oils, vegetable oils, PCL products and lanolin for protection against aqueous solutions, film-forming agents, such as sodium alginate, triethanolamine alginate, polyacrylates, polyvinyl alcohol or cellulose ethers against the effect of organic solvents, or substances based on mineral oils, vegetable oils or silicone oils as “lubricants” against severe mechanical stresses on the skin;
moisturizing substances: the following substances, for example, are used as moisture-controlling agents (moisturizers): sodium lactate, urea, alcohols, sorbitol, glycerol, propylene glycol, collagen, elastin or hyaluronic acid;
active ingredients having a keratoplastic effect: benzoyl peroxide, retinoic acid, colloidal sulfur and resorcinol;
antimicrobial agents, for example triclosan or quaternary ammonium compounds;
oily or oil-soluble vitamins or vitamin derivatives that can be applied dermally: for example vitamin A (getinol in the form of the free acid or derivatives thereof), panthenol, pantothenic acid, folic acid, and combinations thereof, vitamin E (tocopherol), F; essential fatty acids; or niacinamide (nicotinic acid amide);
vitamin-based placenta extracts: active ingredient compositions comprising especially vitamins A, C, E, B₂₁, B₁₂, folic acid and biotin, amino acids and enzymes as well as compounds of the trace elements magnesium, silicon, phosphorus, calcium, manganese, iron or copper;
skin repair complexes: obtainable from inactivated and disintegrated cultures of bacteria of the bifidus group;
plants and plant extracts: for example amica, aloe, beard lichen, ivy, stinging nettle, ginseng, henna, camomile, marigold, rosemary, sage, horsetail or thyme;
animal extracts: for example royal jelly, propolis, proteins or thymus extracts;
cosmetic oils that can be applied dermally: neutral oils of the Miglyol 812 type, apricot kernel oil, avocado oil, babassu oil, cottonseed oil, borage oil, thistle oil, groundnut oil, gamma-oryzanol, rosehip-seed oil, hemp oil, hazelnut oil, blackcurrant-seed oil, jojoba oil, cherry-stone oil, salmon oil, linseed oil, cornseed oil, macadamia nut oil, almond oil, evening primrose oil, mink oil, olive oil, pecan nut oil, peach kernel oil, pistachio nut oil, rape oil, rice-seed oil, castor oil, safflower oil, sesame oil, soybean oil, sunflower oil, tea tree oil, grapeseed oil or wheatgerm oil.

The preparations in stick form are preferably anhydrous but may in certain cases comprise a certain amount of water which, however, in general does not exceed 40% by weight, based on the total weight of the cosmetic preparation.

If the cosmetic preparations and formulations according to the invention are in the form of semi-solid products, that is to say in the form of ointments or creams, they may likewise be anhydrous or aqueous. Such preparations and formulations are, for example, mascaras, eyeliners, foundations, make-up for the cheeks, eyeshadows, or compositions for treating rings under the eyes.

If, on the other hand, such ointments or creams are aqueous, they are especially emulsions of the water-in-oil type or of the oil-in-water type that comprise, apart from the pigment, from 1 to 98.8% by weight of the fatty phase, from 1 to 98.8% by weight of the aqueous phase and from 0.2 to 30% by weight of an emulsifier.

Such ointments and creams may also comprise further conventional additives, for example perfumes, antioxidants, preservatives, gel-forming agents, UV filters, colourants, pigments, pearlescent agents, undyed polymers as well as inorganic or organic fillers.

If the preparations are in the form of a powder, they consist essentially of a mineral or inorganic or organic filler, for example, talc, zinc stearate, mica, kaolin, nylon powders (in particular Orgasol), polyethylene powders, Teflon, starch, boron nitride, microspheres of copolymers, such as Expancel (Nobel Industrie), Polytrap (Dow Corning), silicone resin microbeads (Tospeard from Toshiba, for example), polyethylene powder or polyamide powder, as well as adjuvants such as binders, colourants, etc.

The fillers may be present in an amount ranging from 0 to 35% of the total weight of the composition, preferably from 0.5 to 15%.

Such preparations may likewise comprise various adjuvants conventionally employed in cosmetics, such as perfumes, antioxidants, preservatives, etc.

If the cosmetic preparations and formulations according to the invention are nail varnishes, they consist essentially of nitrocellulose and a natural or synthetic polymer in the form of a solution in a solvent system, it being possible for the solution to comprise other adjuvants, for example pearlescent agents.

In that embodiment, the dyed polymer is present in an amount of approximately from 0.1 to 5% by weight.

The cosmetic preparations and formulations according to the invention may also be used for colouring the hair, in which case they are used in the form of shampoos, creams or gels that are composed of the basic substances conventionally employed in the cosmetics industry and comprise at least one pigment of formula as defined above.

The cosmetic preparations and formulations according to the invention are prepared in the conventional manner, for example by mixing or stirring the components together, optionally with heating, so that the mixtures melt.

The composition examples below are given by way of illustration and without a limiting nature.

The Examples which follow serve to illustrate the invention without limiting it thereto. Parts are parts by weight and percentages are percentages by weight. Temperatures are given in degrees Celsius.

The pigments of formula (I) as defined in Table 1 are used for the following formulation examples.

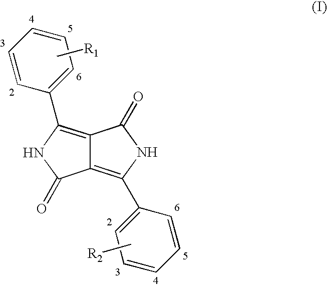

TABLE 1

| Pig- | | |
|---|---|---|
| ment | R₁ | R₂ |
| 1 | (4)-CH₃ | (4)-CH₃ |
| 2 | (3)-CH₃ | (3)-CH₃ |
| 3 | (4)-Cl | (4)-Cl |
| 4 | (4)-Br | (4)-Br |
| 5 | H | (4)-CH₃ |
| 6 | (4)-OCH₃ | (4)-OCH₃ |
| 7 | (4)-F | (4)-F |
| 8 | (3)-OCH₃ | (3)-OCH₃ |
| 9 | (3)-Br | (3)-Br |
| 10 | (4)-OCH₂CH₃ | (4)-OCH₂CH₃ |
| 11 | (3)-Cl | (3)-Cl |
| 12 | (4)-C(CH₃)₃ | (4)-C(CH₃)₃ |
| 14 | H | (4)-CN |
| 15 | H | (4)-OCH₃ |
| 16 | (3)-Cl | (4)-Cl |
| 17 | (4)-Cl | (4)-OCH₃ |
| 18 | (4)-Cl | (4)-CH₃ |
| 19 | 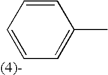 | (4)-Cl |
| 20 | (4)-CH(CH₃)₂ | (4)-CH(CH₃)₂ |
| 21 | (2)-Cl | (2)-Cl |
| 22 | H | (3)-Cl |
| 23 | H | (2)-Cl |
| 24 | H | (4)-Br |
| 25 | H | (3)-Br |
| 26 | H | (4)-F |
| 27 | H | (4)-C(CH₃)₃ |
| 28 | H | (2)-CH₃ |
| 29 | H | (3)-OCH₃ |
| 30 | 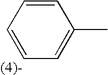 | H |
| 31 | H | (4)-CH(CH₃)₂ |
| 32 | (4)-Cl | (3)-CH₃ |
| 33 | (4)-Cl | (3)-Br |
| 34 | (4)-Cl | (4)-C(CH₃)₃ |
| 35 | (4)-OCH₃ | (4)-CH₃ |
| 36 | (2)-OCH₃ | (2)-OCH₃ |
| 37 | H | (3)-CH₃ |
| 38 | (3)-Cl | (4)-CH₃ |
| 39 | (3)-CH₃ | (4)-CH₃ |
| 40 | (2)-CH₃ | (2)-CH₃ |
| 41 | H | (2)-OCH₃ |
| 42 | (4)-Cl | (3)-OCH₃ |
| 43 | (4)-Cl | (4)-F |
| 44 | (3)-F | (3)-F |
| 45 | H | (3)-F |
| 46 | (4)-Cl | (4)-Br |
| 47 | (4)-C(CH₃)₃ | (3)-CH₃ |
| 48 | (4)-S(CH₂)₉CH₃ | (4)-S(CH₂)₉CH₃ |
| 49 | (4)-Cl | (2)-Cl |
| 50 | H | (4)-OCH(CH₃)₂ |
| 51 | (4)-Cl | (3)-OCH(CH₃)₂ |
| 52 | 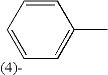 | 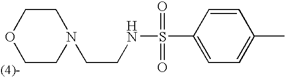 |

All the above mentioned pigments are prepared according to known methods, whereby the specific surface is controlled by a proper selection of the method and the corresponding setup or reaction parameters of the given method by which the specific surface area could be achieved. Possible methods include for example grinding the crude pigments in the presence or absence of a solvent and where appropriate with the aid of grinding elements. Typical examples of grinding methods well known in the literature are dry grinding, wet grinding and kneading. The crude pigments can also be dissolved and re-precipitated from a solution in an appropriate solvent. Typical examples of solvents are concentrated sulphuric acid (acid pasting) or highly polar solvents with the addition of a small amount of a strong alkali. The solution of the colorant is then precipitated by mixing with water or an appropriate solvent with or without an acid or a base. Where appropriate, a pigment can also be grown to the desired surface area by stirring in a solvent or in an emulsion at a certain temperature (conditioning, Ostwald ripening). In certain cases, the desired surface area can also be achieved by a proper selection of the reaction conditions in the pigment synthesis and the subsequent hydrolysis/work up of the pigment. Generally, methods can also be combined to achieve the desired surface area. Milling and conditioning are also highly specific and have to be selected and developed depending on the individual pigment. References are given for example in WO 03/064541 and WO 02/068541.

### EXAMPLE 1

Cheek Color

| Number | Ingredients | Amount [%] |
|---|---|---|
| 1 | Sericite PHN | 58.67 |
| 2 | Zinc Stearate | 4.55 |
| 3 | Magnesium Carbonate NF Light | 2.00 |
| 4 | Boron Nitride | 5.50 |
| 5 | Methyl Paraben | 0.20 |
| 6 | Red Iron Oxide | 2.44 |
| 7 | Pigment 1 | 1.85 |
| 8 | D&C Red 30 (Talc Blend) | 3.76 |
| 9 | Yellow Iron Oxide | 1.36 |
| 10 | Octyl Palmitate | 5.00 |
| 11 | Colorona Russet | 8.73 |
| 12 | Timica Sparkle | 4.77 |
| 13 | Duochrome RY | 1.17 |

Ingredients 1-9 are added and mixed uniformly.

Ingredient 10 is sprayed into the phase and mixed until the phase is completely wetted out and uniform.

Ingredients 11-13 are added and the composition is mixed until uniform. Afterwards the mixture is passed through a micropulverizer.

A red cheek color having excellent in-use properties is obtained in that manner.

### EXAMPLE 2

Cheek Color

| Number | Ingredients | Amount [%] |
|---|---|---|
| 1 | Alpine Talc 141 | 60.80 |
| 2 | Zinc Stearate | 5.00 |
| 3 | Kaolin | 2.00 |
| 4 | Boron Nitride | 5.00 |
| 5 | Methyl Paraben | 0.20 |
| 6 | Pigment 1 | 2.21 |
| 7 | FD&C Yellow 5 Aluminum | 0.57 |
| 8 | Ultramarine Violet | 1.46 |
| 9 | Manganese Violet | 3.73 |
| 10 | Octyl Palmitate | 5.00 |
| 11 | Cloisonne Gold | 7.75 |
| 12 | Cloisonne Red | 4.48 |
| 13 | Flamenco Superpearl 100 | 1.80 |

Ingredients 1-9 are added and mixed uniformly. Ingredient 10 is sprayed into the phase and mixed until the phase is completely wetted out and uniform. Ingredients 11 and 12 are added and the composition is mixed until uniform. Afterwards the mixture is passed through a micropulverizer.

A red cheek color having excellent in-use properties is obtained in that manner.

### EXAMPLE 3

Powder Eyeshadow

| Number | Ingredients | Amount [%] |
|---|---|---|
| 1 | Alpine Talc 141 | 57.58 |
| 2 | Zinc Stearate | 5.26 |
| 3 | Boron Nitride | 5.26 |
| 4 | Propyl Paraben | 0.32 |
| 5 | Sericite PHN | 10.53 |
| 6 | Pigment 1 | 2.63 |
| 7 | Ferric Ferrocyanide | 0.53 |
| 8 | Octyl Palmitate | 5.26 |
| 9 | Duochrome BR | 12.63 |

Ingredients 1-7 are added and mixed uniformly. Ingredient 8 is sprayed into the phase and mixed until the phase is completely wetted out and uniform. Ingredient 9 is added and the composition is mixed until uniform. Afterwards the mixture is passed through a micropulverizer.

Powder eyeshadow having excellent in-use properties is obtained in that manner.

### EXAMPLE 4

Eyeshadow Formulation

| Number | Ingredients | Amount [%] |
|---|---|---|
| 1 | Sericite PHN | 57.59 |
| 2 | Zinc Stearate | 5.26 |
| 3 | Boron Nitride | 5.26 |
| 4 | Propyl Paraben | 0.32 |
| 5 | Wet Ground Mica PGM-3 | 5.26 |
| 6 | Nylon-12 | 5.26 |
| 7 | Pigment 1 | 2.63 |
| 8 | Ferric Ferrocyanide | 0.53 |
| 9 | Octyl Palmitate | 5.26 |
| 10 | Duochrome BR | 12.63 |

The formulation is prepared in analogy of Example 3.

A powder eyeshadow having excellent in-use properties is obtained in that manner.

### EXAMPLE 5

A waterproof eyeshadow cream having excellent in-use properties
has the following composition:

| Number | Ingredients | Amount [%] |
|---|---|---|
| 1 | acrylic acid/butyl acrylate/methyl | 10.00 |
| | methacrylate copolymer, 30% | |
| | emulsion | |
| 2 | Pigment 1 | 10.00 |
| 3 | mineral oil | 8.50 |
| 4 | glycerol | 5.50 |
| 5 | microcrystalline wax | 3.00 |
| 6 | stearic acid | 3.00 |
| 7 | ultramarine blue | 2.00 |
| 8 | sorbitan monostearate | 1.50 |
| 9 | TEA | 1.50 |
| 10 | lanolin | 1.00 |
| 11 | methyl-hydroxypropylcellulose | 0.50 |
| 12 | preservative | qs |
| 13 | water | to 100 |

### EXAMPLE 6

A waterproof eyeshadow gel having excellent in-use properties
has the following composition:

| Number | Ingredients | Amount [%] |
|---|---|---|
| 1 | propylene glycol | 5.00 |
| 2 | sucrose distearate | 3.00 |
| 3 | isopropyl palmitate | 3.00 |
| 4 | mineral oil | 3.00 |
| 5 | lanolin oil | 2.00 |
| 6 | synthetic hectorite | 2.00 |
| 7 | di-Na-EDTA | 0.02 |
| 8 | Pigment 1 | 1.00 |
| 9 | CI Pigment Blue 15 | 0.50 |
| 10 | preservative | qs |
| 11 | water | to 100 |

### EXAMPLE 7

Face powder

| Number | Ingredients | Amount [%] |
|---|---|---|
| 1 | Alpine Talc 141 | 33.97 |
| 2 | Wet Ground Mica PGM-3 | 5.00 |
| 3 | Zinc Stearate | 5.00 |
| 4 | Nylon-12 | 3.00 |
| 5 | Aluminum Starch Octenylsuccinate | 25.00 |
| 6 | Boron Nitride | 2.00 |
| 7 | Silica | 10.00 |
| 8 | Methyl Paraben | 0.25 |
| 9 | Propyl Paraben | 0.10 |
| 10 | Imidazolidinyl Urea | 0.30 |
| 11 | Magnesium Carbonate NF Light | 1.00 |
| 12 | TiO₂ pigment | 6.00 |
| 13 | Pigment 1 | 0.08 |
| 14 | Yellow Iron Oxide | 1.10 |
| 15 | Black Iron Oxide | 0.10 |
| 16 | Octyl Palmitate | 3.00 |
| 17 | Lanolin Oil | 3.00 |
| 18 | Tocopheryl Acetate | 0.10 |
| 19 | Mineral Oil | 1.00 |

Ingredients 1-15 are added and mixed uniformly. The mixture is passed through a micropulverizer. After mixing, ingredients 16-19 are sprayed into the phase and mixed until the phase is completely wetted out and uniform. Afterwards the mixture is passed through a micropulverizer.

A face powder having excellent in-use properties is obtained in that manner.

### EXAMPLE 8

Powder Foundation

| Number | Ingredients | Amount [%] |
|---|---|---|
| 1 | Alpine Talc 141 | 50.3 |
| 2 | Sericite PHN | 21.71 |
| 3 | Zinc Stearate | 6.12 |
| 4 | Kaolin | 1.02 |
| 5 | Silica | 1.02 |
| 6 | Boron Nitride | 3.32 |
| 7 | Methyl Paraben | 0.31 |
| 8 | Magnesium Carbonate NF Light | 1.53 |
| 9 | Pigment 1 | 1.08 |
| 10 | Black Iron Oxide | 0.56 |
| 11 | Yellow Iron Oxide | 7.03 |
| 12 | TiO₂ pigment | 0.90 |
| 13 | Octyl Palmitate | 4.08 |
| 14 | Lanolin Alcohol | 1.02 |

Ingredients 1-12 are added and mixed uniformly. The mixture is passed through a micropulverizer. After mixing at elevated temperature, ingredients 13 and 14 are sprayed into the phase and mixed until the phase is completely wetted out and uniform. Afterwards the mixture is passed through a micropulverizer.

A powder foundation having excellent in-use properties is obtained in that manner.

### EXAMPLE 9

Face Powder

| Number | Ingredients | Amount [%] |
|---|---|---|
| 1 | Alpine Talc 141 | 75.8 |
| 2 | Sericite PHN | 9.62 |
| 3 | Zinc Stearate | 5.15 |
| 4 | Magnesium Carbonate NF Light | 0.10 |
| 5 | Nylon-12 | 1.01 |
| 6 | Silica | 1.01 |
| 7 | Pigment 1 | 0.84 |
| 8 | Yellow Iron Oxide | 2.53 |
| 9 | Black Iron Oxide | 0.51 |
| 10 | Methyl Paraben | 0.20 |
| 11 | Propyl Paraben | 0.10 |
| 12 | Octyl Palmitate | 1.01 |
| 13 | Mineral Oil | 1.01 |
| 14 | Dimethicone | 1.01 |
| 15 | Tocopheryl Acetate | 0.10 |

Ingredients 1-11 are added and mixed uniformly. The mixture is passed through a micropulverizer. After mixing, ingredients 12-15 are sprayed into the phase and mixed until the phase is completely wetted out and uniform. Afterwards the mixture is passed through a micropulverizer.

A face powder having excellent in-use properties is obtained in that manner.

### EXAMPLE 10

A foundation of the following composition is used:

| Number | Ingredients | Amount [%] |
|---|---|---|
| 1 | Cutina KD 16 | 0.80 |
| 2 | Cutina FS 45 | 1.40 |
| 3 | Lanette 16 | 1.00 |
| 4 | Arlacel 60 | 0.20 |
| 5 | paraffin oil pearl | 8.00 |
| 6 | isopropyl stearate | 6.00 |
| 7 | Myritol 318 | 4.00 |
| 8 | Softisan 100 | 2.00 |
| 9 | Abil 100 | 0.20 |
| 10 | Controx KS | 0.05 |
| 11 | Uniphen P 23 | 1.00 |
| 12 | talcum Pharma G | 5.00 |
| 13 | titanium white | 6.00 |
| 14 | Pigment 1 | 1.50 |
| 15 | demineralized H₂O | 56.10 |
| 16 | propylene carbonate | 0.10 |
| 17 | Veegum ultra | 0.80 |
| 18 | glycerol 87% | 5.00 |
| 19 | Natrosol 250 HHR | 0.30 |
| 20 | TEA C, pure | 0.55 |

Substances 1-11 are melted together, and substance 12 is dispersed in that mixture. The mixture is then heated to from 75 to 80° C.

Separately therefrom, substances 15 and 16 are mixed, and substance 17 is dispersed homogeneously in that mixture. Substance 19 is then distributed homogeneously in that mixture; once the increase in swelling has ceased, substance 18 is stirred in and the whole mixture is heated to from 75 to 80° C.

The second mixture is then added to the first mixture, with intensive stirring, substance 20 is then stirred in homogeneously, and the resulting emulsion is stirred until it has cooled to room temperature. Substances 13 and 14 are then dispersed in by means of a dissolver, and the resulting make-up is then passed through a triple roller.

There is obtained a red make-up having excellent in-use properties and an intense bright red colour of outstanding fastness to light.

### EXAMPLE 11

A powder foundation of the following composition is used

| | | |
|---|---|---|
| Number | Ingredients | Amount [%] |

Phase A

| | | |
|---|---|---|
| 1 | Talc | 48.20 |
| 2 | Mica and Methicone | 34.00 |
| | (Toshiki Sericite OS-61D) | |
| 3 | Pigment 1 | 5.00 |
| 4 | Kaolin | 6.00 |
| 5 | Zinc Stearate | 3.00 |
| 6 | Methyl Paraben | 0.20 |
| 7 | Propyl Paraben | 0.10 |

Phase B

| | | |
|---|---|---|
| 8 | Dicaprylyl Maleate | 3.00 |
| 9 | PEG-400 Diisostearate | 0.50 |

Phase A is put into high shear mixer and mixed until color are completely extended. All ingredients of phase B are put together and mixed until phase B is fully homogenous.

Phase B is sprayed to phase A with high mixing.

The united phases are mixed fully homogenous by a high shear mixer.

There is obtained a powder foundation having excellent in-use properties and an intense bright red colour of outstanding fastness to light.

### EXAMPLE 12

Lipstick Formulation

| Number | Ingredients | Amount [%] |
|---|---|---|
| 1 | Castor Oil LISP | 15.00 |
| 2 | Pigment 1 | 1.40 |
| 3 | FD&C Blue 1 B3016 Aluminum Lake | 0.25 |
| 4 | FD&C Yellow 5 B3014 Aluminum Lake | 0.20 |
| 5 | TiO₂ pigment | 3.00 |
| 6 | Cosmetic Yellow C33-8073 | 1.00 |
| 7 | Red Iron Oxide 3080 | 3.20 |
| 8 | Castor Oil LISP | 31.40 |
| 9 | White Beeswax | 2.00 |
| 10 | Performalene 400 | 4.00 |
| 11 | Camauba Wax | 2.00 |
| 12 | Candelilla Wax | 5.00 |
| 13 | Caprylic/Capric Triglyceride | 8.00 |
| 14 | Octyl Methoxycinnamate | 7.50 |
| 15 | Lanolin Oil | 2.00 |
| 16 | Stearyl Alcohol | 2.00 |
| 17 | Jojoba Oil | 6.00 |
| 18 | Shea Butter | 2.00 |
| 19 | Cetyl Palmitate | 3.00 |
| 20 | Propyl Paraben | 0.20 |
| 21 | Tocopheryl Acetate | 0.10 |
| 22 | Lipstick Fragrance | 0.75 |

Ingredients 8-21 are mixed at a temperature of 75-80° C. until the phase is uniform. Ingredients 1-7 are mixed together and grinded in a ball mill or 3-roll mill. Afterwards ingredients 1-7 are added to the mixture of ingredients 8-21. The mixture is mixed at a temperature of 75-80° C. Afterwards Ingredient 22 is added and the mixture is mixed at a temperature of about 70° C.

A lipstick having excellent in-use properties is obtained in that manner.

### EXAMPLE 13

Lipstick Formulation

| Number | Ingredients | Amount [%] |
|---|---|---|
| 1 | Ozokerite Wax | 4.02 |
| 2 | Camauba Wax | 3.76 |
| 3 | Candelilla Wax | 4.74 |
| 4 | White Beeswax | 7.98 |
| 5 | Myristyl Lactate | 5.37 |
| 6 | Octyl Palmitate | 3.63 |
| 7 | Shea Butter | 1.00 |
| 8 | Isopropyl Palmitate | 3.45 |
| 9 | Castor Oil USP | 41.62 |
| 10 | Propyl Paraben | 0.20 |
| 11 | Isopropyl Isostearate | 2.55 |
| 12 | Castor Oil USP | 10.00 |
| 13 | Pigment 1 | 2.74 |
| 14 | TiO₂ pigment | 3.22 |
| 15 | Red Iron Oxide 3080 | 1.23 |
| 16 | FD&C Yellow 5 B3014 Aluminum Lake | 1.78 |
| 17 | FD&C Blue 1 B3016 Aluminum Lake | 0.21 |
| 18 | Cloisonne Gold | 2.00 |
| 19 | Lipstick Fragrance | 0.50 |

Ingredients 1-11 are mixed at a temperature of 75-80° C. until the phase is uniform. Ingredients 12-18 are mixed together and grinded in a ball mill or 3-roll mill. Afterwards ingredients 12-18 are added to the mixture of ingredients 1-11. The mixture is mixed at a temperature of 75-80° C. Afterwards Ingredient 19 is added and the mixture is mixed at a temperature of about 70° C.

A lipstick having excellent in-use properties is obtained in that manner.

### EXAMPLE 14

A lipstick base of the following composition is used:

| Number | Ingredients | Amount [%] |
|---|---|---|
| 1 | cera alba | 11.40 |
| 2 | candelilla wax | 8.10 |
| 3 | carnauba wax | 3.80 |
| 4 | Lunacera M | 6.00 |
| 5 | castor oil | 38.80 |
| 6 | Controx KS | 0.10 |
| 7 | aromatic oil | 1.00 |
| 8 | Amerlate P | 2.50 |
| 9 | OH Ian | 1.60 |
| 10 | isopropyl palmitate | 10.10 |
| 11 | Dow Corning 556 | 2.80 |
| 12 | Dow Corning 1401 | 3.30 |
| 13 | TiO₂ pigment | 2.30 |
| 14 | Pigment 1 | 8.20 |

Substances 8-10 are mixed together, and substances 13 and 14 are dispersed in that mixture. The resulting paste is then passed several times through a triple roller.

In the meantime, substances 1-6 are melted and stirred together homogeneously, and then substances 7, 11 and 12 are stirred in.

The two mixtures are then mixed together while hot until homogeneous distribution is achieved. The hot mass is then poured into a lipstick mould and allowed to cool.

There are obtained lipsticks having an intense bright red colour of outstanding fastness to light and very good gloss.

### EXAMPLE 15

A water-in-oil lipstick emulsion having the following composition
is prepared analogously to Example 14:

| Number | Ingredients | Amount [%] |
|---|---|---|
| 1 | mineral oil | 28.50 |
| 2 | glycerol bis(2-heptylundecanoate) | 28.50 |
| 3 | Pigment 1 | 9.20 |
| 4 | polyethylene wax | 10.00 |
| 5 | candelilla wax | 10.00 |
| 6 | ceresin wax | 5.7 |
| 7 | water | 3.00 |
| 8 | glycerol | 2.00 |
| 9 | carnauba wax | 1.40 |
| 10 | castor oil | 1.45 |
| 11 | magnesium aluminium silicate | 0.15 |
| 12 | benzyldimethylstearylammonium chloride | 0.05 |
| 13 | hydrogenated lecithin | 0.05 |

There are obtained lipsticks having an intense bright red colour of outstanding fastness to light and very good gloss.

### EXAMPLE 16

An oil-in-water lipstick emulsion having the following composition is
prepared analogously to Example 14:

| Number | Ingredients | Amount [%] |
|---|---|---|
| 1 | glycerol tri-2-ethylhexanoate | 31.80 |
| 2 | jojoba oil | 20.00 |
| 3 | ceresin wax | 10.00 |
| 4 | castor oil | 10.00 |
| 5 | Pigment 1 | 10.00 |
| 6 | lanolin oil | 5.00 |
| 7 | water | 5.00 |
| 8 | microcrystalline wax | 3.00 |
| 9 | canauba wax | 2.00 |
| 10 | surface-active substances based on alkyl ethers | 2.00 |
| 11 | glycerol | 1.00 |
| 12 | polyvinyl alcohol | 0.20 |

There are obtained lipsticks having an intense bright red colour of outstanding fastness to light and very good gloss.

### EXAMPLE 17

A non-greasy lipstick having the following composition is
prepared analogously to Example 14:

| Number | Ingredients | Amount [%] |
|---|---|---|
| 1 | white beeswax | 20.00 |
| 2 | ozocerite | 10.00 |
| 3 | Pigment 1 | 9.00 |
| 4 | anhydrous lanolin | 5.00 |
| 5 | propylene glycol recinoleate | 4.00 |
| 6 | liquid paraffin | 3.00 |
| 7 | isopropyl myristate | 3.00 |
| 8 | carnauba wax | 2.50 |
| 9 | cetyl alcohol | 2.00 |
| 10 | CI Pigment Blue 15 | 1.00 |
| 11 | castor oil | 40.50 |

There are obtained lipsticks having an intense bright red colour of outstanding fastness to light and very good gloss.

### EXAMPLE 18

A transfer-resistant lipstick having the following composition is
prepared analogously to Example 14:

| Number | Ingredients | Amount [%] |
|---|---|---|
| 1 | cyclomethicone | 50.00 |
| 2 | isodecane | 12.00 |
| 3 | Pigment 1 | 8.00 |
| 4 | synthetic wax | 7.20 |
| 5 | isostearyltrimethylpropane-siloxy silicate | 6.00 |
| 6 | cetyl stearate/acetylated lanolin, 90:10 | 6.00 |
| 7 | ceresin | 4.80 |
| 8 | paraffin | 3.60 |
| 9 | TiO₂ pigment | 2.00 |
| 10 | methylparaben | 0.30 |
| 11 | propylparaben | 0.10 |

There are obtained lipsticks having an intense bright red colour of outstanding fastness to light and very good gloss.

### EXAMPLE 19

Liquid Make-up Formulation

| Number | Ingredients | Amount [%] |
|---|---|---|
| 1 | Deionized Water | 50.46 |
| 2 | Magnesium Aluminum Silicate | 2.06 |
| 3 | Carboxy Methyl Cellulose | 0.1 |
| 4 | Lecithin | 0.10 |
| 5 | Methyl Paraben | 0.31 |
| 6 | Imidazolidinyl Urea | 0.52 |
| 7 | Butylene Glycol | 5.16 |
| 8 | Triethanolamine 99% | 2.06 |
| 9 | Kaolin | 2.06 |
| 10 | TiO₂ pigment | 7.73 |
| 11 | Yellow Iron Oxide | 5.46 |
| 12 | Pigment 1 | 1.55 |
| 13 | Black Iron Oxide | 0.46 |
| 14 | Alpine Talc 141 | 2.06 |
| 15 | Stearic Acid | 6.19 |
| 16 | Glyceryl Monostearate Pure | 2.58 |
| 17 | Isopropyl Lanolate | 2.06 |
| 18 | Lanolin Alcohol | 0.21 |
| 19 | Mineral Oil | 8.25 |
| 20 | Propyl Paraben | 0.10 |
| 21 | Makeup Fragrance | 0.52 |

Ingredients 1-4 are mixed and the ingredients 5-14 are added to the phase and the composition is mixed until completely uniform. The mixture is milled using either a colloid mill or ball mill. This composition is mixed at a temperature of about 75° C.

Ingredients 15-20 are mixed and heated up to 75° C. Afterwards the oil phase is slowly added to the water phase with continuous mixing. After cooling to 50° C. ingredient 21 is added and the composition is mixed until completely uniform.

A liquid make-up formulation having excellent in-use properties is obtained in that manner.

### EXAMPLE 20

Make-up Formulation

| Number | Ingredients | Amount [%] |
|---|---|---|
| 1 | Deionized Water | 61.21 |
| 2 | Butylene Glycol | 8.00 |
| 3 | Xantham Gum | 0.34 |
| 4 | Magnesium Aluminum Silicate | 0.51 |
| 5 | Imidazolidinyl Urea | 0.25 |
| 6 | Methyl Paraben | 0.30 |
| 7 | Triethanolamine 99% | 1.31 |
| 8 | Silica | 2.50 |
| 9 | TiO₂ pigment | 5.10 |
| 10 | Pigment 1 | 0.15 |
| 11 | Yellow Iron Oxide | 0.90 |
| 12 | Black Iron Oxide | 0.05 |
| 13 | Cetyl Ethylhexanoate | 3.54 |
| 14 | Decyl Oleate | 3.54 |
| 15 | C₁₂₋₁₅ Alkyl Benzoate | 2.53 |
| 16 | Stearic Acid | 3.54 |
| 17 | Isostearic Acid | 1.01 |
| 18 | Cetyl Alcohol | 0.51 |
| 19 | Caprylic/Capric Triglyceride | 1.01 |
| 20 | Propyl Paraben | 0.15 |
| 21 | BHT | 0.05 |
| 22 | Dimethicone | 3.50 |

Ingredients 1-4 are mixed and the ingredients 5-12 are added to the phase and the composition is mixed until completely uniform. The mixture is milled using either a colloid mill or ball mill. This composition is mixed at a temperature of about 75° C.

Ingredients 13-22 are mixed and heated up to 75° C. Afterwards the oil phase is slowly added to the water phase with continuous mixing.

A make-up formulation having excellent in-use properties is obtained in that manner.

### EXAMPLE 21

A make-up stick having excellent in-use properties has the following
composition:

| Number | Ingredients | Amount [%] |
|---|---|---|
| 1 | mineral oil and lanolin alcohol | 22.50 |
| 2 | Laneth-5 | 15.00 |
| 3 | TiO₂ pigment | 11.00 |
| 4 | cetyl alcohol | 5.00 |
| 5 | carnauba wax | 4.50 |
| 6 | Pigment 1 | 4.00 |
| 7 | yellow iron oxide | 4.00 |
| 8 | candilla wax | 0.50 |
| 9 | perfume and preservative | qs |
| 10 | oleyl alcohol | to 100 |

### EXAMPLE 22

A blusher (powder) having excellent in-use properties has the following
composition:

| Number | Ingredients | Amount [%] |
|---|---|---|
| 1 | talcum | 58.00 |
| 2 | zinc stearate | 15.00 |
| 3 | rice starch | 15.00 |
| 4 | Pigment 1 | 12.00 |
| 5 | perfume | q.s. |

### EXAMPLE 23

The following substances are used for a nail varnish:

| Number | Ingredients | Amount [%] |
|---|---|---|
| 1 | sodium selenite | 0.01 |
| 2 | ethyl acetate | 20.00 |
| 3 | isobutyl acetate | 26.99 |
| 4 | isopropyl alcohol | 2.00 |
| 5 | toluene | 20.00 |
| 6 | nitrocellulose | 17.00 |
| 7 | saccharose acetate isobutyrate | 8.00 |
| 8 | dibutyl phthalate | 3.80 |
| 9 | 1,3-butylene glycol | 0.20 |
| 10 | Pigment 1 | 1.00 |
| 11 | stearylalkonium hectorite | 1.00 |

A red nail varnish having very good in-use properties and outstanding gloss is obtained. After application of the nail varnish, a waiting period of three days and removal of the nail varnish, it is found that the nail has remained completely unstained.

### EXAMPLE 24

The following substances are used for a water-based nail varnish:

| Number | Ingredients | Amount [%] |
|---|---|---|
| 1 | demineralized water | 58.85 |
| 2 | TiO₂ pigment | 5.60 |
| 3 | Pigment 1 | 2.16 |
| 4 | talcum | 5.72 |
| 5 | potassium cetyl phosphate | 1.50 |
| 6 | propylene glycol | 8.00 |
| 7 | Mg—Al silicate | 1.00 |
| 8 | cellulose gum (high viscosity) | 0.14 |
| 9 | ester of saccharose and coconut fatty acid | 0.20 |
| 10 | methylparaben | 0.20 |
| 11 | EDTA | 0.05 |
| 12 | propylene glycol dicaprylate/dicaprate | 10.80 |
| 13 | isostearyl-stearyl stearate | 2.00 |
| 14 | sorbitan monolaurate | 3.00 |
| 15 | cetyl alcohol | 0.50 |
| 16 | propylparaben | 0.10 |
| 17 | DMDM-hydantoin | 0.18 |

A red nail varnish having very good in-use properties and outstanding gloss is obtained. After application of the nail varnish, a waiting period of three days and removal of the nail varnish, it is found that the nail has remained completely unstained.

### EXAMPLE 25

The following substances are used for a mascara formulation:

| Number | Ingredients | Amount [%] |
|---|---|---|
| 1 | stearic acid | 3.50 |
| 2 | glycerol stearate | 6.00 |
| 3 | beeswax | 7.00 |
| 4 | propylparaben | 0.10 |
| 5 | demineralized water | 38.25 |
| 6 | methylparaben | 0.10 |
| 7 | polyvinylpyrrolidone | 6.00 |
| 8 | propylene glycol | 3.00 |
| 9 | sodium carboxymethylcellulose | 0.15 |
| 10 | pigment 1 | 10.40 |
| 11 | kaolin | 3.50 |
| 12 | ethyl acrylate/methyl acrylate (8/2) | 22.00 |

Mixtures of substances 1 to 4 and 5 to 9 are heated separately from one another until homogeneous mixtures are formed; the mixtures are then combined and stirred thoroughly until a homogeneous phase is obtained. Components 10 and 11 are then dispersed in a portion of that phase, and the dispersion is then added to the remainder. Component 12 is then added with stirring.

A mascara formulation having excellent in-use properties is obtained in that manner.

### EXAMPLE 26

A mascara formulation for hair is prepared from
the following components:

| Number | Ingredients | Amount [%] |
|---|---|---|
| 1 | mascara base (mixture of beeswax, carnauba wax, | 15.00 |
| | stearic acid, Ceteareth 25, PEG-2 stearate, | |
| | mineral oil, hydrogenated coconut | |
| | oil and cetyl alcohol) | |
| 2 | dimethicone | 1.50 |
| 3 | preservative | 0.50 |
| 4 | demineralized water | 42.10 |
| 5 | triethanolamine 85% | 0.45 |
| 6 | thickener mixture (xanthan gum, hectorite, | 0.45 |
| | cellulose gum) | |
| 7 | pigment 1 | 10.00 |
| 8 | acrylate copolymer | 30.00 |

Ingredients 1 are heated to approximately 75° C., with slow stirring, in a steel tank. In a separate vessel, ingredient 3 is dissolved in ingredient 4, and ingredient 6 is added in such a manner as to obtain a gel that is homogeneous at room temperature. Ingredients 2 and 5 are then added, and heating to approximately 75° C. is carried out. With moderate stirring, the mixture of ingredients 2, 3, 4, 5 and 6 is added to ingredient 1, and stirring is carried out until the product is homogeneous. Ingredient 7 is dispersed in a portion of that product by means of a three-roll mill, ingredient 8 is added with stirring, and the dispersion is then added to the remainder of the product and mixed thoroughly.

A mascara formulation for hair having good in-use properties is obtained.

### EXAMPLE 27

A water-in-oil mascara having excellent in-use properties
has the following composition:

| Number | Ingredients | Amount [%] |
|---|---|---|
| 1 | polyisobutylene | 57.60 |
| 2 | microcrystalline wax | 20.00 |
| 3 | Pigment 1 | 10.00 |
| 4 | carnauba wax | 7.00 |
| 5 | bentonite | 3.00 |
| 6 | beeswax | 2.00 |
| 7 | lanolin | 0.40 |

### EXAMPLE 28

A hair mascara having the following composition is prepared
analogously to Example 26:

| Number | Ingredients | Amount [%] |
|---|---|---|
| 1 | Pigment 1 | 12.00 |
| 2 | white beeswax | 6.50 |
| 3 | propylene glycol | 6.00 |
| 4 | carnauba wax | 4.25 |
| 5 | cetearyl alcohol and dicetyl phosphate and | 4.00 |
| | ceteth-10 phosphate (Crodafos CES) | |
| 6 | PVP/hydrolysed wheat protein copolymer | 4.00 |
| 7 | Steareth-10 | 1.00 |
| 8 | stearyl alcohol | 1.00 |
| 9 | PVP | 1.00 |
| 10 | Steareth-2 | 0.50 |
| 11 | Laneth-5 | 0.50 |
| 12 | potassium hydroxide | 0.24 |
| 13 | hydroxyethylcellulose | 0.10 |
| 14 | di-Na-EDTA | 0.10 |
| 15 | preservative | qs |
| 16 | water | to 100 |

A mascara formulation for hair having good in-use properties is obtained.

### EXAMPLE 29

A foundation cream having excellent in-use properties has the following
composition:

| Number | Ingredients | Amount [%] |
|---|---|---|
| 1 | TiO₂ pigment | 12.79 |
| 2 | oleyl alcohol | 4.57 |
| 3 | glycerol stearate | 3.65 |
| 4 | propylene glycol | 3.65 |
| 5 | stearic acid | 1.83 |
| 6 | magnesium aluminium silicate | 0.91 |
| 7 | triethanolamine 99% | 0.91 |
| 8 | CI Iron Oxide Yellow | 0.64 |
| 9 | Pigment 1 | 0.42 |
| 10 | CI Pigment Brown 6 | 0.37 |
| 11 | carboxymethylcellulose | 0.10 |
| 12 | water | to 100 |

In every formulation Example 1-29, Pigment 1 can be replaced by any of the pigments 2-24, as well as by mixtures of pigments 1-24 as well as by mixtures of pigments 1-24 with one or more other suitable pigments as described above.

1. A cosmetic formulation comprising at least one pigment of formula (i): 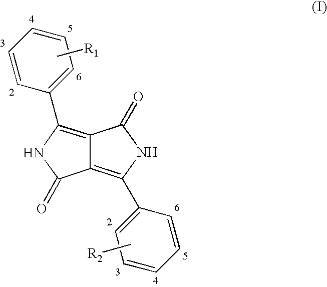
wherein
R₁ signifies hydrogen; SO₃M; linear or branched C₁-C₁₈alkyl, which can be unsubstituted or substituted by one or more halogen, OH, OR₃, SR₃, SO₂NR₃R₄, NH₂, NR₃R₄, COOR₄, CONR₃R₄, OCOR₃ or SO₃M; linear or branched C₁-C₄alkoxy, which can be unsubstituted or substituted by one or more halogen, OH, OR₃, SR₃, SO₂NR₃R₄, NH₂, NR₃R₄, COOR₄, CONR₃R₄, OCOR₃ or SO₃M; halogen; phenyl, which can be unsubstituted or substituted by one or more halogen, OH, OR₃, SR₃, SO₂NR₃R₄, NH₂, NR₃R₄, COOR₄, CONR₃R₄, OCOR₃ or SO₃M,
R₂ signifies hydrogen; SO₃M; linear or branched C₁-C₁₈alkyl, which can be unsubstituted or substituted by one or more halogen, OH, OR₃, SR₃, SO₂NR₃R₄, NH₂, NR₃R₄, COOR₄, CONR₃R₄, OCOR₃ or SO₃M; linear or branched C₁-C₄alkoxy, which can be unsubstituted or substituted by one or more halogen, OH, OR₃, SR₃, SO₂NR₃R₄, NH₂, NR₃R₄, COOR₄, CONR₃R₄, OCOR₃ or SO₃M; halogen; phenyl, which is substituted by one or more halogen, OH, OR₃, SR₃, SO₂NR₃R₄, NH₂, NR₃R₄, COOR₄, CONR₃R₄, OCOR₃ or SO₃M,
wherein
R₃ signifies linear or branched C₁-C₃₀alkyl; C₃-C₃₀-alkenyl; C₃-C₁₂cycloalkyl; C₆-C₁₄aryl, which can be unsubstituted or substituted by one or more C₁-C₆alkyl, C₅-C₆cycloalkyl, C₁-C₆alkoxy, C₁-C₆thioalkyl or halogen; C₇-C₂₄aralkyl, which can be unsubstituted or substituted by one or more C₁-C₆alkyl, C₅-C₆cycloalkyl, C₁-C₆alkoxy, C₁-C₆thioalkyl or halogen or C₈-C₂₄aralkenyl, which can be unsubstituted or substituted by one or more C₁-C₆alkyl, C₅-C₆cycloalkyl, C₁-C₆alkoxy, C₁-C₆thioalkyl or halogen; or C₁-C₄alkylene-C₄-C₈cylcoalkyl, wherein the ring contains at least one heteroatom selected from the group of N, O or S,
R₄ signifies hydrogen; linear or branched C₁-C₃₀alkyl; C₃-C₃₀-alkenyl; C₃-C₁₂cycloalkyl; C₆-C₁₄aryl, which can be unsubstituted or substituted by one or more C₁-C₆alkyl, C₅-C₆cycloalkyl, C₁-C₆alkoxy, C₁-C₆thioalkyl or halogen; C₇-C₂₄aralkyl, which can be unsubstituted or substituted by one or more C₁-C₆alkyl, C₅-C₆cycloalkyl, C₁-C₆alkoxy, C₁-C₆thioalkyl or halogen or C₈-C₂₄aralkenyl, which can be unsubstituted or substituted by one or more C₁-C₆alkyl, C₅-C₆cycloalkyl, C₁-C₆alkoxy, C₁-C₆thioalkyl or halogen; or C₁-C₄alkylene-C₄-C₈cylcoalkyl, wherein the ring contains at least one heteroatom selected from the group of N, O or S, and
M signifies hydrogen; a metal atom; or an unsubstituted or substituted ammonium group,
wherein the pigments have a specific surface area (BET) of 15-200 m²/g, and with the proviso that if
(i) R₁ is H or Cl in 4 position, then R₂ is not H or Cl in 4 position.

2. A cosmetic formulation according to claim 1, wherein the pigments have a specific surface area (BET) of 25-200 m²/g.

3. A cosmetic formulation according to claim 1, wherein the pigments have a specific surface area (BET) of 15-170 m²/g.

4. A cosmetic formulation according to claim 1, wherein the pigments have a specific surface area (BET) of 20-150 m²/g.

5. A cosmetic formulation according to claim 1, wherein the pigments have a specific surface area (BET) of 25-150 m²/g.

6. A cosmetic formulation according to claim 1, wherein
R₁ signifies hydrogen; linear or branched C₁-C₁₂alkyl, which can be unsubstituted or substituted by one or more halogen, OH, OR₃, SR₃, SO₂NR₃R₄, NH₂, NR₃R₄, COOR₄, CONR₃R₄, OCOR₃ or SO₃M; linear or branched C₁-C₄alkoxy, which can be unsubstituted or substituted by one or more halogen, OH, OR₃, SR₃, NH₂, SO₂NR₃R₄, NR₃R₄, COOR₄, CONR₃R₄, OCOR₃ or SO₃M; halogen; phenyl, which can be unsubstituted or substituted by one or more halogen, OH, OR₃, SR₃, SO₂NR₃R₄, NH₂, NR₃R₄, COOR₄, CONR₃R₄, OCOR₃ or SO₃M, wherein R₃, R₄ and M have the same meanings as defined above.

7. A cosmetic formulation according to claim 1, wherein R₂ signifies hydrogen; linear or branched C₁-C₁₂alkyl, which can be unsubstituted or substituted by one or more halogen, OH, OR₃, SR₃, SO₂NR₃R₄, NH₂, NR₃R₄, COOR₄, CONR₃R₄, OCOR₃ or SO₃M; linear or branched C₁-C₄alkoxy, which can be unsubstituted or substituted by one or more halogen, OH, OR₃, SR₃, SO₂NR₃R₄, NH₂, NR₃R₄, COOR₄, CONR₃R₄, OCOR₃ or SO₃M; halogen; phenyl, which is substituted by one or more halogen, OH, OR₃, SR₃, NH₂, NR₃R₄, COOR₄, CONR₃R₄, OCOR₃ or SO₃M,
wherein R₃, R₄ and M have the same meanings as defined above in claim 1.

8. A cosmetic formulation according to claim 7, wherein
R₃ signifies linear or branched C₁-C₁₈alkyl; C₃-C₁₈-alkenyl; C₃-C₈cycloalkyl; C₆-C₈aryl, which can be unsubstituted or substituted by one or more C₁-C₄alkyl, C₅-C₆cycloalkyl, C₁-C₄alkoxy, C₁-C₄thioalkyl or halogen; C₇-C₁₈aralkyl, which can be unsubstituted or substituted by one or more C₁-C₄alkyl, C₅-C₆cycloalkyl, C₁-C₄alkoxy, C₁-C₄thioalkyl or halogen or C₈-C₁₈aralkenyl, which can be unsubstituted or substituted by one or more C₁-C₄alkyl, C₅-C₆cycloalkyl, C₁-C₄alkoxy, C₁-C₄thioalkyl or halogen; or C₁-C₄alkylene-C₄-C₈cylcoalkyl, wherein the ring contains at least one heteroatom selected from the group of N, O or S,
R₄ signifies hydrogen; linear or branched C₁-C₁₈alkyl; C₃-C₁₈-alkenyl; C₃-C₈cycloalkyl; C₆-C₈aryl, which can be unsubstituted or substituted by one or more C₁-C₄alkyl, C₅-C₆cycloalkyl, C₁-C₄alkoxy, C₁-C₄thioalkyl or halogen; C₇-C₁₈aralkyl, which can be unsubstituted or substituted by one or more C₁-C₄alkyl, C₅-C₆cycloalkyl, C₁-C₄alkoxy, C₁-C₄thioalkyl or halogen or C₈-C₁₈aralkenyl, which can be unsubstituted or substituted by one or more C₁-C₄alkyl, C₅-C₆cycloalkyl, C₁-C₄alkoxy, C₁-C₄thioalkyl or halogen; or C₁-C₄alkylene-C₄-C₈cylcoalkyl, wherein the ring contains at least one heteroatom selected from the group of N, O or S, and
M signifies hydrogen; a metal atom; or an unsubstituted or substituted ammonium group.

9. A cosmetic formulation according to claim 1, wherein
R₁ signifies hydrogen; linear or branched C₁-C₆alkyl, which can be unsubstituted or substituted by one or more OR₃, SR₃, SO₂NR₃R₄, NR₃R₄ or COOR₄; linear or branched C₁-C₄alkoxy, which can be unsubstituted or substituted by one or more OR₃, SR₃, SO₂NR₃R₄, NR₃R₄ or COOR₄; halogen; phenyl, which can be unsubstituted or substituted by one or more OR₃, SR₃, SO₂NR₃R₄, NR₃R₄ or COOR₄,
R₂ signifies hydrogen; linear or branched C₁-C₆alkyl, which can be unsubstituted or substituted by one or more OR₃, SR₃, SO₂NR₃R₄, NR₃R₄ or COOR₄; linear or branched C₁-C₄alkoxy, which can be unsubstituted or substituted by one or more OR₃, SR₃, SO₂NR₃R₄, NR₃R₄ or COOR₄; halogen; phenyl, which is substituted by one or more OR₃, SR₃, SO₂NR₃R₄, NR₃R₄ or COOR₄, wherein
R₃ signifies linear or branched C₁-C₈alkyl; C₃-C₈-alkenyl; C₃-C₈cycloalkyl; C₆-C₈aryl, which can be unsubstituted or substituted by one or more C₁-C₄alkyl, C₅-C₆cycloalkyl, C₁-C₄alkoxy, C₁-C₄thioalkyl or halogen; C₇-C₁₂aralkyl, which can be unsubstituted or substituted by one or more C₁-C₄alkyl, C₅-C₆cycloalkyl, C₁-C₄alkoxy, C₁-C₄thioalkyl or halogen or C₈-C₁₂aralkenyl, which can be unsubstituted or substituted by one or more C₁-C₄alkyl, C₅-C₆cycloalkyl, C₁-C₄alkoxy, C₁-C₄thioalkyl or halogen; C₁-C₄alkylene-morpholino; or C₁-C₄alkylene-piperidino and
R₄ signifies hydrogen; linear or branched C₁-C₈alkyl; C₃-C₈-alkenyl; C₃-C₈cycloalkyl; C₆-C₈aryl, which can be unsubstituted or substituted by one or more C₁-C₄alkyl, C₅-C₆cycloalkyl, C₁-C₄alkoxy, C₁-C₄thioalkyl or halogen; C₇-C₁₂aralkyl, which can be unsubstituted or substituted by one or more C₁-C₄alkyl, C₅-C₆cycloalkyl, C₁-C₄alkoxy, C₁-C₄thioalkyl or halogen or C₈-C₁₂aralkenyl, which can be unsubstituted or substituted by one or more C₁-C₄alkyl, C₅-C₆cycloalkyl, C₁-C₄alkoxy, C₁-C₄thioalkyl or halogen; C₁-C₄alkylene-morpholino; or C₁-C₄alkylene-piperidino.

10. A cosmetic formulation according to claim 1 comprising
a) from 0.0001 to 50% by weight, based on the total weight of the preparation, of at least one pigment of formula (I), and
b) from 50 to 99.9999% by weight, based on the total weight of the preparation, of a cosmetically suitable carrier.

11. A cosmetic preparation or formulation according to claim 1, which is in the form of a stick comprising up to 99.9999% by weight of fatty components.

12. A cosmetic preparation or formulation according to claim 1, which is in the form of an anhydrous or aqueous ointment or cream.

13. A cosmetic preparation or formulation according to claim 1, which is in the form of a water-in-oil emulsion or in the form of an oil-in-water emulsion comprising from 1 to 98.8% by weight of the fatty phase, from 1 to 98.8% by weight of the aqueous phase and from 0.2 to 30% by weight of an emulsifier, in each case based on the total weight.

14. A cosmetic preparation or formulation according to claim 1, which is in the form of a powder and comprises an inorganic or organic filler.

15. A cosmetic preparation or formulation according to claim 1, which is in the form of a nail varnish and comprises from 0.1 to 5% by weight of the pigment in a varnish base.

16. A cosmetic preparation or formulation according to claim 1, which is in the form of a shampoo, a cream or a gel for colouring the hair that is composed of the basic substances conventionally employed in the cosmetics industry.

17. A cosmetic preparation or formulation according to claim 1, which additionally comprises conventional cosmetic constituents.

18. A cosmetic preparation or formulation according to claim 14, wherein the inorganic or organic filler is talc, zinc stearate, mica, kaolin, nylon powders, polyethylene powders, Teflon, starch, boron nitride, microspheres of copolymers, silicone resin microbeads, polyethylene powder or polyamide powder, and the adjuvants are binders or colorants.

19. A cosmetic preparation or formulation according to claim 1, wherein the conventional cosmetic constituents are selected from perfumes, antioxidants, preservatives and UV filters.
